(19) United States
(12) Patent Application Publication
(10) Pub. No.: US 2023/0061046 A1
(43) Pub. Date: Mar. 2, 2023

(54) SYSTEMS AND METHODS FOR RESPONSIVE ULTRASOUND STIMULATION FOR IMMUNO-MODULATION TREATMENT

(71) Applicant: REGENTS OF THE UNIVERSITY OF MINNESOTA, MINNEAPOLIS, MN (US)

(72) Inventors: Hubert Lim, Minneapolis, MN (US); Daniel Zachs, Minneapolis, MN (US); Claire Kaiser, Minneapolis, MN (US); Rachel Graham, Minneapolis, MN (US)

(73) Assignee: REGENTS OF THE UNIVERSITY OF MINNESOTA, MINNEAPOLIS, MN (US)

(21) Appl. No.: 17/794,098

(22) PCT Filed: Jan. 22, 2021
(86) PCT No.: PCT/US2021/014583

§ 371 Date: Jul. 20, 2022

Related U.S. Application Data
(60) Provisional application No. 62/964,493, filed on Jan. 22, 2020.

(51) Int. Cl. A61N 7/00; A61B 5/00; G16H 40/63

(52) U.S. Cl. CPC A61N 7/00; A61B 5/4836; A61B 5/416; G16H 40/63; A61N 2007/0086; A61N 2007/0078; A61B 5/14503

(57) ABSTRACT

Systems and methods are provided for ultrasound stimulation for immunomodulation treatment. Ultrasound stimulation of the spleen may be used to alter immune and thereby inflammatory responses of a subject by modulating specific biomarkers or cytokines associated with inflammation. A closed-loop or responsive ultrasound stimulation of the spleen may be implemented by tracking biomarkers in the blood that indicate when the stimulation is working. Modulation of specific cytokines and/or erythrocyte sedimentation rate may be performed in response to ultrasound stimulation in a diseased state.

### CROSS-REFERENCE TO RELATED APPLICATIONS

This application claims the benefit of U.S. Provisional Patent Application Ser. No. 62/964,493 filed on Jan. 22, 2020 and entitled “Systems and Methods for Responsive Ultrasound Stimulation for Immuno-Modulation Treatment,” which is incorporated herein by reference as if set forth in its entirety for all purposes.

### STATEMENT REGARDING FEDERALLY SPONSORED RESEARCH

This invention was made with government support under N660011824018 awarded by Department of Defense. The government has certain rights in the invention.

### BACKGROUND

Human rheumatoid arthritis (RA) is an autoimmune disorder that prominently features inflammatory changes in the peripheral joints. The disease affects more than 24 million people, worldwide. Common life-altering symptoms in patients with active RA include joint stiffness, tenderness, swelling, and reduced range of motion, as well as constitutional symptoms such as fatigue and fever.

Recent advances in understanding of cytokine networks and the molecular basis of lymphocyte interactions have led to important novel therapies in the past 20 years. Unfortunately, current therapies provide inadequate control of inflammatory symptoms in rheumatoid arthritis patients or can lead to unfavorable side effects, as also can occur across multiple other acute and chronic inflammatory disorders or conditions, such as inflammatory bowel disease, fatty liver disease, atherosclerosis, sepsis, infections and some types of cancers or responses to cancer treatments. Corticosteroids are effective in managing active RA or inflammatory symptoms, but their chronic use is attended by high risk of short and long-term side-effects.

The cholinergic anti-inflammatory pathway (CAP) has been extensively studied because of its role in modulating the mammalian immune response. This pathway relies on a robust neural-immune interaction in which peripheral nerves communicate with and can alter the activity of the immune system. The mechanism of action has been studied in multiple animal studies showing that in response to infection or injury, the parasympathetic vagus nerve transmits signals from the brain to the adrenergic splenic nerve, which interacts with splenic immune cells. When the vagus nerve is experimentally stimulated with electrical current, this neural-immune reflex is triggered, dampening the inflammatory response to infection or tissue injury. This pathway requires the interaction of the vagus nerve, splenic nerve, spleen and splenocytes. Vagus nerve stimulation (VNS) has been shown to reduce in vivo cytokine production during endotoxemia in rat and mouse models. VNS has also been used to treat arthritis in animal models, and there is a reported link between the cholinergic nervous system and the inflammatory process in inflamed joints. More recently, VNS was used to treat rheumatoid arthritis in human patients using implantable vagus nerve electrode cuffs.

These effects have also been achieved through direct electrical stimulation of different splenic nerves entering the spleen. Electrical stimulation of the apical splenic nerves and the arterial splenic nerves (located more basally along the spleen) also achieve suppression of TNF. However, when using various knock-out mice lacking T cells, it is clear that the apical nerves are achieving an anti-inflammatory effect that is different from the vagus nerve and arterial splenic nerve pathways.

There remains a need for non-invasive, non-toxic and cost-effective alternatives to immunomodulatory anti-inflammatory drugs. There also remains a need for a treatment for inflammatory conditions with a greater level of control of the immune response and with greater specificity. Such a system may provide for treatment for previously challenging inflammation conditions, such as recovery from stroke, spinal cord injury and acute kidney failure. These solutions are also urgently needed to improve standard of care in rheumatoid arthritis sufferers and other acute and chronic inflammatory conditions listed above.

### SUMMARY OF THE DISCLOSURE

The present disclosure addresses the aforementioned drawbacks by providing a system and method for responsive or closed-loop ultrasound stimulation for immuno-modulation treatment. Ultrasound stimulation of the spleen, in accordance with the present disclosure, may be used to alter immune and thereby inflammatory responses of a subject by modulating specific biomarkers or cytokines associated with inflammation. In some configurations, a closed-loop or responsive ultrasound stimulation of the spleen may be implemented by tracking biomarkers in the blood that indicate when the stimulation is working, and thus it is possible to titrate the ultrasound stimulation to reach a certain treatment state.

In one configuration, a method is provided for ultrasound stimulation of a subject. The method includes applying ultrasound stimulation to a target of the subject with initial ultrasound stimulation parameters. The method also includes receiving feedback data from a sensor, where the feedback data are measured by the sensor and are indicative of biomarkers being modulated by the applied ultrasound stimulation. The method also includes adjusting the initial ultrasound stimulation parameters based upon the received feedback data to create adjusted ultrasound stimulation parameters. Ultrasound stimulation may be applied to the target of the subject with the adjusted ultrasound stimulation parameters to treat an inflammatory condition of the subject.

In one configuration, a system is provided for ultrasound stimulation of a subject. The system includes a feedback sensor implanted within a subject. The system also includes a computer system configured to: i) apply ultrasound stimulation to a target of the subject with initial ultrasound stimulation parameters; ii) receive feedback data from a sensor, where the feedback data are measured by the sensor and are indicative of biomarkers being modulated by the applied ultrasound stimulation; iii) adjust the initial ultrasound stimulation parameters based upon the received feedback data to create adjusted ultrasound stimulation parameters; and iv) apply ultrasound stimulation to the target of the subject with the adjusted ultrasound stimulation parameters to treat an inflammatory condition of the subject.

In one configuration, a method is provided for ultrasound stimulation of a subject. The method includes determining a target for the ultrasound stimulation of the subject and determining ultrasound stimulation parameters. The method also includes applying ultrasound stimulation to the target of the subject with the ultrasound stimulation parameters to treat an inflammatory condition of the subject and modulate biomarkers in the treatment. The biomarkers include a combination of one or more of erythrocyte sedimentation rate (ESR), tumor necrosis factor TNF-α, interleukin IL-6, and interleukin IL-10.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a non-limiting example flowchart for delivering ultrasound stimulation to a subject with a closed-loop system.

FIG. 2 is a block diagram of an ultrasound system that may be used in accordance with the systems and methods of the present disclosure.

FIG. 3 is a block diagram of a non-limiting example device for providing stimulation.

FIG. 4A is a profile view of a non-limiting example therapy module housing.

FIG. 4B is a profile view of a non-limiting example therapy module base.

FIG. 4C is a profile view of a non-limiting example assembled therapy module.

FIG. 5 is a block diagram for a non-limiting example system configuration.

FIG. 6 is a graph of non-limiting example results of data showing for the first time in humans with an inflammatory health condition the ability to modulate inflammation and biomarkers by applying ultrasound to the spleen for treatment in accordance with the present disclosure.

### DETAILED DESCRIPTION

Systems and methods are provided for ultrasound stimulation for immunomodulation treatment. Ultrasound stimulation of the spleen, in accordance with the present disclosure, may be used to alter immune and thereby inflammatory responses of a subject by modulating specific biomarkers or cytokines associated with inflammation. In some configurations, a closed-loop or responsive ultrasound stimulation of the spleen may be implemented by tracking biomarkers in the blood that indicate when the stimulation is working, and thus it is possible to titrate the ultrasound stimulation to reach a certain state. Modulation of specific cytokines and erythrocyte sedimentation rate (ESR; measures of inflammation in the body) may be performed in response to ultrasound of the spleen in a diseased state. In non-limiting examples, a disease state may include an inflammatory condition, including rheumatoid arthritis, or neural or tissue injury, and the like, or the system and methods may be used with immune compromised subjects. Ultrasound stimulation of the spleen may also be used to modulate biomarkers associated with the immune response.

Noninvasive ultrasound (US) energy may be delivered to the abdomen to generate diminished inflammation and tissue damage during renal ischemic reperfusion injury (IRI). These anti-inflammatory effects may be mediated by the spleen. Ultrasound stimulation may activate the same cholinergic anti-inflammatory pathway triggered by vagus nerve stimulation (VNS). Splenocyte transfer studies indicated that the leukocytes harvested from US-treated spleens could confer protection from IRI when injected into naïve recipient mice. US may transform these splenocytes to an anti-inflammatory state and thus the US stimulation approach may be used to prevent or treat inflammatory conditions in addition to renal IRI.

Ultrasound may also be delivered indirectly to stimulate an implanted device, which would in turn provide stimulation to the spleen. The implanted device may include an implantable sensor to provide for controlling the device, and which may detect cytokine biomarkers.

A closed-loop wearable system may be used to deliver ultrasound stimulation targeting the spleen that is adjusted based on simultaneous imaging. The integrated closed-loop system may integrate advanced ultrasound phased array beamforming stimulation technology with advanced ultrasound imaging technology to create an integrated wearable system. US stimulation of the spleen provides the ability to modulate and suppress the immune response directly in human subjects.

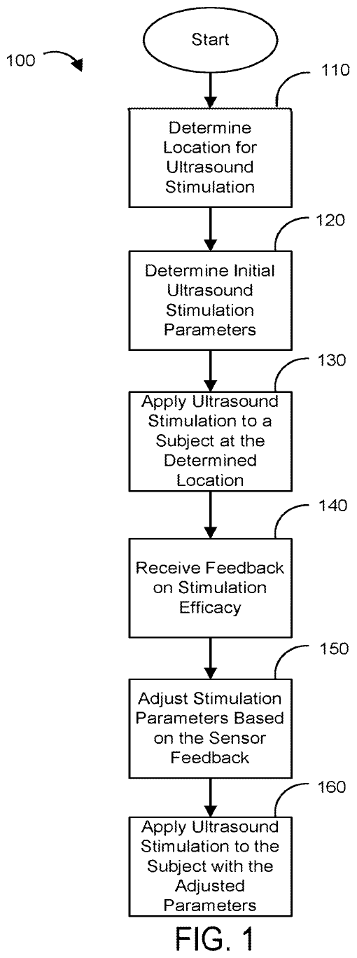

Referring to FIG. 1, a flowchart is provided depicting non-limiting example steps for a method 100 for closed-loop immunomodulation ultrasound stimulation. A location for ultrasound stimulation may be determined at step 110. Initial ultrasound stimulation parameters for creating a desired immunomodulation effect may be determined at step 120. Ultrasound stimulation of the determined target location may then be delivered at step 130. Feedback from a sensor may be used to gauge the efficacy of the stimulation in creating the desired immunomodulation effect at step 140. The stimulation parameters may be adjusted to optimize the desired immunomodulation effect at step 150. Optimized ultrasound stimulation may be applied to the subject with the adjusted parameters at step 160.

The spleen may be selected as the target location for ultrasound stimulation in step 110. Stimulation of the spleen, or cells or elements within the spleen, may be performed without specifically stimulating neurons or neural elements in the spleen, which has been a target of previous techniques. In some configurations, ultrasound stimulation may target activating the terminals of neurons or the receptors located within the synapses between the neuron and cells in the spleen, or modulating non-neuronal cells in the spleen may be performed to cause therapeutic effects (e.g., immune cells or other structures in the spleen that alter the immune cell function). Ultrasound stimulation of the spleen causes a decrease in erythrocyte sedimentation rate (ESR) and thus indicates inflammation is going down. Also, select cytokines may be created or altered through ultrasound stimulation of the spleen, such as tumor necrosis factor (TNF), such as TNF-α, and interleukins (IL), such as IL-6 and IL-10. Ultrasound stimulation may also cause a reduction in a number of swollen joints. To allow the inflammation to go back up to a certain level, the ultrasound stimulation can be periodically turned off; and thus, the immune response can be adjusted up and down based on pulsing or periodic ultrasound patterns. In some configurations, stimulation of non-splenic targets may be used, such as other organs, lymph nodes, peripheral nerves/ganglion, spinal cord, receptors, and the like, which also enables stimulation of multiple neuroimmune pathways.

Biomarkers may be modulated individually or in any combination. The biomarkers may include blood markers, erythrocyte sedimentation rate (ESR), CRP, ferritin/d-dimer, tumor necrosis factor TNF-α, interleukin IL-6, interleukin IL-10, and the like. This list includes the first set of biomarkers to have been discovered and shown to be systematically modulated for ultrasound stimulation of the spleen in human patients with an inflammatory condition. The ability to treat inflammatory conditions by modulating these biomarkers with ultrasound stimulation of the spleen provides for a non-invasive, non-toxic and cost-effective alternative to immunomodulatory anti-inflammatory drugs, and also provides a treatment for inflammatory conditions with a greater level of control of the immune response and with greater specificity.

Initial ultrasound stimulation parameters and delivery of the initial stimulation in steps 120 and 130 may be performed based upon the stimulation parameters used to generate the above cytokines and immunomodulation. Parameters may also be identified as treating inflammatory arthritis. Non-limiting example stimulation parameters include stimulation between 50 kHz to 7 MHz, such as between 500 kHz to 1 MHz, such as stimulation at 1 MHz, and pressures in a range of 100 kPa to 500 kPa or 25 kPa to 10 MPa depending on the stimulation frequency. In some configurations, approximately 350 kPa may result in an efficacious effect. Additional non-limiting example parameters include duty cycles of 10-50%, 50-100%, or a 100% duty cycle. Stimulation may be moved around the spleen, such as continuously moving a stimulation around the spleen for a specified period of time. In a non-limiting example, stimulation may be moved around the spleen for 30 minutes. In some configurations, stimulating different parts of the spleen periodically may be effective such that lower duty cycles may be selected. In some configurations, the initial ultrasound stimulation parameters include at least one of stimulation in the range of greater than or equal to 500 kHz to less than or equal to 1 MHz; pressures in a range of greater than or equal to 100 kPa to less than or equal to 500 kPa; or duty cycles of greater than or equal to 16% to less than or equal to 50%. Non-limiting example stimulation parameters are included in Table 1 below.

TABLE 1
non-limiting example stimulation parameters.

| Parameter | Example 1 Range | Example 2 Range |
|---|---|---|
| Center Frequency | 100 kHz-7 MHz | 500 kHz-1 MHz |
| Pulse Repetition | Stimulation on: 50 micros- | 350 ms on/650 ms off |
| Rate (PRF) | 2 sec | 500 ms on/500 ms off |
| | Stimulation off: 1 ms-10 sec | 1 sec on/6 sec off |
| Treatment | 1 min-1 hr sessions | 2 min-30 min sessions |
| Duration | | |
| Pressure at | 25 kPa-10 MPa | 100 kPa-500 kPa |
| Target Area | | |
| Duty Cycle | See Pulse Repetition Rate | 16%-50% |

| | |
|---|---|
| Focus/Targeting | Full organ or sub-organ area |
| Imaging | Ultrasound imaging techniques, non-limiting example |

In some aspects of the present disclosure, a combination of TNF-α, IL-6 and IL-10 together with ESR can be used to titrate ultrasound stimulation of the spleen. These biomarkers and parameters may be used to track the stimulation and recovery from inflammatory conditions, such as arthritis, and the like.

In some configurations at step 140, data from a blood draw may be used to acquire cytokine information. Blood samples can be taken and then the results may be inputted into a hospital/patient data server. In non-limiting examples, the hospital/patient data server may then connect via wireless to the patient's device or through the patients' own internet connection to the hospital/patient data server to upload those values, or the user/fitter can input those values into the wearable device controller. Once the updated values have been entered, the system may then optimize/adjust the stimulation parameters.

In some configurations at step 140, data from a sensor may be used to provide feedback for a closed-loop system able to deliver stimulation in an automated fashion to prevent an identified level of inflammation, or to generate a desired clinical effect. In a non-limiting example, the sensor is a cytokine sensor that may be implanted into a vascular system of a subject.

In some configurations at step 140, data from genetic expression may be used alone or in combination with cytokine information for feedback in a closed-loop configuration. A non-limiting example description for genetic expression data has been described in: “First-in-human demonstration of splenic ultrasound stimulation for non-invasively controlling inflammation,” Rachel Stegeman Graham, et. al, doi: https://doi.org/10.1101/2020.07.14.20153528, which is hereby incorporated by reference.

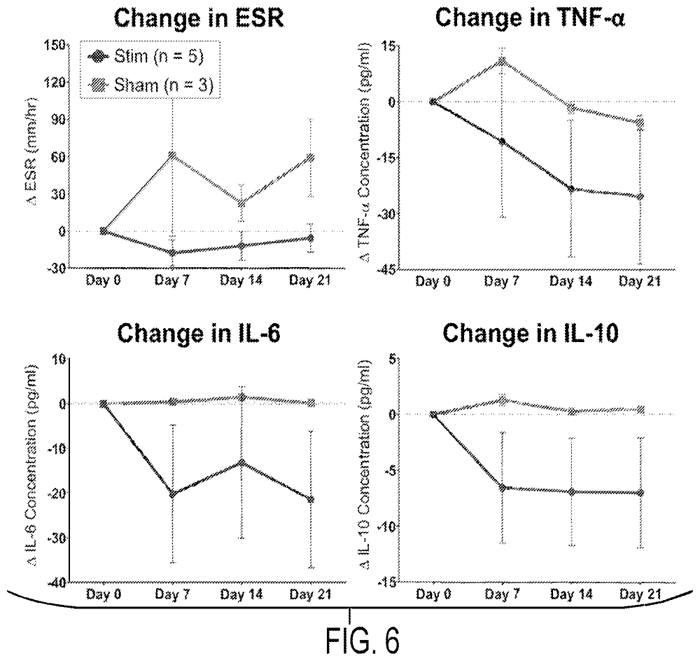

Inflammatory effects may occur over days and may plateau at about 7-14 days, such as depicted in the non-limiting example shown in FIG. 6. Blood samples or sensor feedback, such as from an implantable or minimally invasive sensor, may provide feedback data for readings every day, continuously, or at select times. Stimulation may then be delivered daily for a selected period of time, such as for 30 minutes per day in a non-limiting example, and stimulation may be discontinued when the sensor feedback data values reach a plateau or a certain target level. When the values start to change again, then stimulation may resume to stay at the desired level.

Adjusting the stimulation parameters at step 150 may include accounting for the data input from different biomarkers indicated by the feedback sensor. In some configurations, parameters such as TNF-α may continue to decrease with stimulation over the course of treatment, such that it may not experience a plateau effect as quickly or as much as other parameters, such as IL-6 and IL-10. The method may account for such differences by including a balance of stopping stimulation when there is a period or desired level between the plateau effects of IL-6 and IL-10 versus TNF-α data values. The duty cycle or duration of stimulation may also be adjusted. In one non-limiting example, the duty cycle may be selected to be less initially and to increase over time to drive these biomarkers and then reduce them per day. In another non-limiting example, stimulation may be performed every day for select duty cycle parameters to increase or decrease total energy applied to the spleen.

In some configurations, the adjusted ultrasound stimulation parameters include at least one of stimulation in the range of greater than or equal to 100 kHz to less than or equal to 7 MHz; pressures in a range of greater than or equal to 25 kPa to less than or equal to 10 MPa; stimulation on time of greater than or equal to 50 microseconds to less than or equal to 2 seconds; or stimulation off time of greater than or equal to 1 ms to less than or equal to 10 s.

In some configurations, a wearable device may be used with method 100. The system may be worn each day, and a user may measure their blood biomarker levels to provide input to the system. The system may then adjust the stimulation that is applied after receiving the user provided input.

In some configurations, a wearable device may be used where the device is worn more frequently, such as continuously, and automatically senses the biomarkers using a feedback sensor. The system may have the advantage of being able to stimulate more frequently throughout the day, and may do so for selected periods of time, such as in short bursts.

Closed-loop methods may be used to analyze real-time data from the biomarkers to adjust the ongoing treatment regimen automatically to achieve appropriate levels of local and systemic inflammation. In a non-limiting example, closed-loop algorithms include machine learning or artificial intelligence (AI) routines, that provide for automated targeting of stimuli. These closed-loop algorithms may provide for steering the ultrasound beam to the targets based on real-time ultrasound imaging, and the stimulation patterns delivered over time based on sampling of the immune biomarkers at the local injury site provided by the implantable sensor, and the like.

In some configurations of closed loop systems, when inflammatory indicating biomarkers go up or continue to stay high, then stimulation may be increased. Stimulation may be increased by increasing intensity such as by increasing pressure, duration, PRF, duty cycle, and/or focusing an ultrasound beam to a smaller region. A stimulation beam may also be adjusted to simulate another location, such as a location within a select distance of the initial stimulation, if further reduction in inflammation is desired than was achieved at the first location. The adjusted location may be 1-3 cm away from the first location, but may be selected as any appropriate distance away. The stimulation and assessment of the resulting biomarker levels may be continued at any number of locations if the desired decrease in inflammation is not achieved. In some configurations, a threshold may be set to determine when to adjust the location, such as if reduction in inflammation is less than 25%. Any desired threshold level may be determined. In some configurations, a linear equation may be used to determine how far away from the initial location to move a subsequent stimulation site, such as by determining that proportionally 0 to 4 cm away may correspond to 0% reduction to 50% reduction after 0-14 days. Any number of days may be considered, such as 2 days, or 4 days, or 7 days, or 14 days, and the like.

If pro-inflammatory markers go to a threshold value, such as approximately 50%, then the intensity of the stimulation may be titrated down in a proportional, linear, or exponential way. In a non-limiting example, when 50% to 100% reduction has been achieved, then intensity may be reduced from an initial level down to 0%. In a non-limiting example of an exponential titration, the intensity of the stimulation may be reduced slowly, then drop more quickly in the form of an inverse exponential. This form of reduction may be used when a rapid shut down of inflammation is desired initially, while at the same time recognizing that there may still be a need for some inflammation in the body, so as to slow reduction of intensity of stimulation initially and then rapidly towards 0 when inflammation levels have become low.

In some configurations, biomarker assessment may be performed daily for a determined number of days, such as 0-14 days. As shown in FIG. 6, the different biomarkers plateau at different time points that can vary from 7 to 14 days, such that biomarker assessment may be most effective to adjust the ultrasound stimulation parameters during that time period, and depending on the differences in plateau effect or change in ratios of those biomarkers. The plateau effect may exist between the two earliest time points of data collection shown in FIG. 6, which would be between 0 to 7 days, and thus an average of 3.5 days. Therefore, biomarker assessment may be performed every 3.5 days up to 14 days, and even up to 21 days based on FIG. 7. Any number of days may be considered, such as 2 days, 4 days, 7 days, and the like. Biomarker assessment may monitor for a plateau effect in the days a subject is monitored.

The physiological balance between pro- and anti-inflammatory signaling molecules is used by the body to respond to infection and disease, without causing excess inflammatory damage. An imbalance of this balance between pro- and anti-inflammatory cytokines is often a marker of chronic inflammatory diseases and autoimmunity. The systems and methods in accordance with the present disclosure may analyze the detected concentration ratios of specific cytokines, and tailor treatment based on those ratios. The change in cytokines may be detected over time to determine if more or less treatment is required.

Ratios of biomarkers may be used to assess stimulation efficacy, and/or to adjust stimulation as needed to achieve the desired reduction in inflammation. IL-6 is an inflammatory cytokine secreted by macrophages in response to pathogens or viral infections, or can be produced in response to other pro-inflammatory cytokines. IL-6 also stimulates inflammatory pathways in many auto-immune diseases. Conversely, IL-10 is an anti-inflammatory cytokine produced by monocytes and lymphocytes that helps control the immune response by downregulating pro-inflammatory signaling. Assessing the real-time IL-6 to IL-10 ratio may be used as a factor to modulate the ultrasound treatment.

TNF-α and ESR may be modulated using ultrasound stimulation of the spleen. TNF-α is a proinflammatory cytokine released by macrophages to alert other immune cells of an infection or other inflammatory event. It is also elevated in a variety of acute and chronic diseases. ESR, the rate of erythrocyte sedimentation, is a biomarker that is used to measure inflammation. When an inflammatory process exists in the body, it increases the ESR. Measuring these markers of systemic inflammation, TNF-α, ESR or the ratio of TNF-α/ESR, may be used as another factor to modulate the ultrasound treatment. In a non-limiting example, the ratio of IL-6 to TNF-α may be used.

These biomarkers, alone or in combination with others detected by a real-time feedback system, may be analyzed by algorithms or methods in accordance with the present disclosure to automatically adjust the treatment. In addition to those already stated, C-reactive protein (CRP) is an indicator of inflammation, Interferon gamma (IFN-y) is an inflammatory molecule that responds to infection, and Interleukin 1 beta (IL-1p) is an inflammatory cytokine known to produce fever. A feedback system may analyze the real-time ratios of these and other biomarkers, including IL-6/ESR, IL-6/TNF-α, TNF-α/IL-10, ESR/IL-10, ESR/CRP, IL-6/IFN-γ, IL-113/IL-10, and the like to automatically determine the dose of treatment required.

In a non-limiting example, a change in IL6 may be divided by a change in IL10. This may be performed over a range of days, such as a change from day 0 to day 3.5 of IL6 divided by a change in IL10 from day 0 to day 3.5. Any range of days may be used, such as a range from 0 to day 7 for the ratios. If the biomarkers goes higher, which means the pro-inflammatory IL-6 is rising faster than the anti-inflammatory IL-10, the stimulation can be adjusted to better reduce inflammation, such as by changing location for the stimulation, or by changing stimulation parameters. In a non-limiting example, the slope of change of IL-6 vs IL-10 may be used, such as the slope from day 0 to day 7 to adjust stimulation. If the slope is greater for that ratio slope, then a stimulation may be increased in intensity or a shift in location may be performed. If inflammation goes down, or reaches a determined threshold of reduction, then the intensity of stimulation may be reduced.

A non-limiting example algorithm of using ratios to adjust treatment in an automated logic tree is as follows:

```
$If \frac{IL 6 ( CurrentValue )}{IL 10 ( CurrentValue )} \leq 0.3 \times \frac{IL 6 ( 24 hrs - prior )}{IL 10 ( 24 hrs - prior )} ,$
  Then OutputUltrasoundIntensity = 0%
$Else If \frac{IL 6 ( CurrentValue )}{IL 10 ( CurrentValue )} \leq 0.5 \times \frac{IL 6 ( 24 hrs - prior )}{IL 10 ( 24 hrs - prior )} ,$
  Then OutputUltrasoundIntensity = 25%
$Else If \frac{IL 6 ( CurrentValue )}{IL 10 ( CurrentValue )} \leq 0.7 \times \frac{IL 6 ( 24 hrs - prior )}{IL 10 ( 24 hrs - prior )} ,$
  Then OutputUltrasoundIntensity = 50%
$Else If \frac{IL 6 ( CurrentValue )}{IL 10 ( CurrentValue )} \leq 0.9 \times \frac{IL 6 ( 24 hrs - prior )}{IL 10 ( 24 hrs - prior )} ,$
  Then OutputUltrasoundIntensity = 75%
Else OutputUltrasoundIntensity = 100%
End
```

Where IL6 and IL10 may be sensor-measured concentrations of IL-6 and IL-10 biomarkers, CurrentValue is the most recent value measured, 24 hrs-prior is the value measured 24 hours previously, and OutputUltrasoundIntensity is the dose of ultrasound that will be delivered automatically in the next scheduled pre-programmed treatment session. The ultrasound dose may be modulated by the levels of measured IL-6/IL-10, and this is one example of cytokine ratios that may be used in a logic tree to determine optimal ultrasound dosing based on the measured biomarker feedback system. It is to be appreciated that the time period disclosed is a non-limiting example. Any period of time may be used, such as 6 hours, 12 hours, 2 days, 3.5 days, 4 days, 7 days or 14 days. A time range for receiving feedback on biomarker levels may be from 6 hours up to and including 14 days, or from 6 hours up to and including 7 days. Another time range for receiving feedback on biomarker levels may be from 3.5 days up to and including 14 days, or from 3.5 days up to and including 7 days. Time periods may be from 6 hours up to and including 21 days.

In a non-limiting example, a ratio of IL-6/TNF-α may be used, where TNF-α provides for a more generally systemic inflammation effect. If TNF-α has stabilized but IL-6 is still rising, then something abnormal may be happening or an increase in intensity may be used to counteract inflammation.

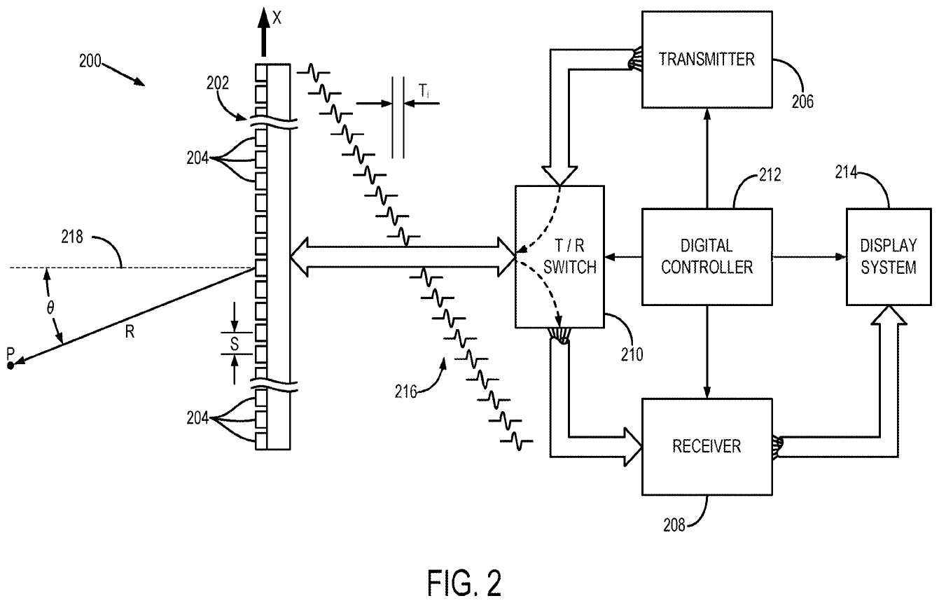

FIG. 2 illustrates an example of an ultrasound system 200 that can be used to implement the methods described in the present disclosure. The ultrasound system 200 includes a transducer array 202 that includes a plurality of separately-driven transducer elements 204. The transducer array 202 can include any suitable ultrasound transducer array, including linear arrays, curved arrays, phased arrays, and so on. Similarly, the transducer array 202 can include a 1D transducer, a 1.5D transducer, a 1.75D transducer, a 2D transducer, a 3D transducer, and so on.

When energized by a transmitter 206, a given transducer element 204 produces a burst of ultrasonic energy. The ultrasonic energy reflected back to the transducer array 202 (e.g., an echo) from the object or subject under study is converted to an electrical signal (e.g., an echo signal) by each transducer element 204 and can be applied separately to a receiver 208 through a set of switches 210. The transmitter 206, receiver 208, and switches 210 are operated under the control of a controller 212, which may include one or more processors. As one example, the controller 212 can include a computer system.

The transmitter 206 can be programmed to transmit unfocused or focused ultrasound waves. In some configurations, the transmitter 206 can also be programmed to transmit diverged waves, spherical waves, cylindrical waves, plane waves, or combinations thereof. Furthermore, the transmitter 206 can be programmed to transmit spatially or temporally encoded pulses.

The receiver 208 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 206 and the receiver 208 can be programmed to implement a high frame rate. For instance, a frame rate associated with an acquisition pulse repetition frequency (“PRF”) of at least 100 Hz can be implemented. In some configurations, the ultrasound system 200 can sample and store at least one hundred ensembles of echo signals in the temporal direction.

The controller 212 can be programmed to implement an imaging sequence using the techniques described in the present disclosure, or as otherwise known in the art. In some embodiments, the controller 212 receives user inputs defining various factors used in the design of the imaging sequence.

A scan can be performed by setting the switches 210 to their transmit position, thereby directing the transmitter 206 to be turned on momentarily to energize transducer elements 204 during a single transmission event according to the designed imaging sequence. The switches 210 can then be set to their receive position and the subsequent echo signals produced by the transducer elements 204 in response to one or more detected echoes are measured and applied to the receiver 208. The separate echo signals from the transducer elements 204 can be combined in the receiver 208 to produce a single echo signal.

In some configurations, ultrasound system 200 may include more than one ultrasound array 202. At least one of the arrays may be used for transmitting ultrasound for therapeutic treatment, and at least one other array may be used to transmit and/or receive ultrasound for imaging. In one non-limiting example, the frequency used for treatment may be lower than the frequency used to produce high-quality images.

The echo signals are communicated to a processing unit (not shown), which may be implemented by a hardware processor and memory, to process echo signals or images generated from echo signals. As a non-limiting example, the processing unit can target the spleen for ultrasound stimulation to treat inflammation using the methods described in the present disclosure. Images produced from the echo signals by the processing unit can be displayed on a display system 214.

In one non-limiting example, a wearable/miniaturized phased array ultrasound device may be used for ultrasound system 200, although other types of ultrasound stimulation devices (e.g. a convention/cart-based US system) may also be used. The wearable device may be placed on a subject and worn for extended periods of time. In some configurations, a reprogrammed smartphone or tablet device may be used to control and/or monitor the stimulation. In one non-limiting example, the reprogrammed smartphone or tablet device may allow for Bluetooth wireless control of the stimulation device. In some configurations, the control and/or monitor system may be an application installed on a smartphone, tablet device, and the like. In another non-limiting example, a number of devices distributed around a subject may be used to provide the ultrasound stimulation. The overall size of a wearable device may be similar to that of other consumer electronic wearable devices, such as smart watches, when using miniaturized ultrasound array technology. In one non-limiting example, the total footprint of a device may be less than 1.2 cm×1.2 cm. In another non-limiting example, the footprint of a device may be less than 40×40 mm. In some non-limiting examples, the total footprint of the device may be larger, such as to cover a larger region of the spleen or other organ. In one non-limiting example, the footprint of the device is the same size as a spleen, which may range from 8-16 cm.

In some configurations, the ultrasound transducer elements 204 can be activated in unison to form a plane wave front. In other configurations, the ultrasound transducer elements 204 can be activated sequentially to form an angled or focused wave front.

In some configurations, beam steering may be used to target selected locations on or in the spleen with ultrasound stimulation. Locations may be selected to optimize the effects of treatment, such as by targeting neural pathways within the spleen. Optimized effects may include modulating the cholinergic anti-inflammatory immune reflex circuit to decrease a subject's inflammation and treat rheumatoid arthritis. This anti-inflammatory reflex pathway involves circuits in the brain (e.g., medulla oblongata C1 neuron activation and hypothalamic-pituitary responses) that project via the vagus nerve to the spleen as well as reciprocal pathways back to the brain, coordinating the immune response of the body.

In some configurations, a wearable energy delivery device will have the capability to image and transmit ultrasound energy to a specified target. Imaging and/or beam steering may also be used to compensate for motion of the spleen, such as changes in depth due to subject motion. The spleen naturally moves in the abdominal cavity during heavy breathing and changes in body position. With imaging feedback, the ultrasound beam focus point can be realigned as the distance to the surface of the spleen changes relative to the transducer array. This provides the potential for closed-loop device operation with the implementation of automated re-targeting algorithms.

To accomplish a beam steered scan, the transmitter 206 imparts a time delay, Tᵢ, to the respective pulses 216 that are applied to successive transducer elements 204. If the time delay is zero, Tᵢ=0, all of the transducer elements 204 will be energized simultaneously and the resulting ultrasonic beam will be directed along an axis 218 normal to the face of the transducer 202 and originating from the center of the transducer array 202. As the time delay increment, Tᵢ, is increased, the ultrasonic beam is directed away from the central axis 218 by an angle, θ. The relationship between the time delay increment, Tᵢ, added successively to each i^{th} signal from one end of the transducer array 202, i=1, to the other end, i=n, is given by the following relationship:

$${T}_{i} = {T}_{0} + ( i - \frac{( n - 1 )}{2} ) \cdot ( \frac{S \cdot sin ( θ )}{c} ) + {( i - \frac{( n - 1 )}{2} )}^{2} \cdot ( \frac{{S}^{2} \cdot cos ( 2 θ )}{2 R c} ) ; \qquad ( 1 )$$

where S is an equal spacing between centers of adjacent transducer elements 204; c is the velocity of sound in the object under study; R is a range, or depth, at which the transmit beam is to be focused; and T₀ is a delay offset that insures that all calculated time delay increment values, Tᵢ, are positive values.

The second term in Eqn. (1) steers the beam to the desired angle, θ, and the third term is employed when the transmitted beam is to be focused at a fixed range, R. A sector scan is performed by progressively changing the time delays, Tᵢ, in successive excitations. In this manner, the angle, θ, is changed in increments to steer the transmitted beam in a succession of directions. When the direction of the beam is above the central axis 218, the timing of the pulses is reversed; however, Eqn. (1) still applies in this situation.

The echo signals produced by each burst of ultrasonic energy emanate from reflecting objects located at successive ranges, or depths, R, along the ultrasonic beam. These are sensed separately by each transducer element 204 in the transducer array 202, and a sample of the magnitude of each echo signal at a particular point in time represents the amount of reflection occurring at a specific range, R. Due to the differences in the propagation paths between a focal point, P, and each transducer element 204, however, these echo signals will not occur simultaneously and their amplitudes will not be equal. A function of the receiver 208 is to amplify and demodulate these separate echo signals, impart the proper time delay to each, and sum them together to provide a single echo signal that accurately indicates the total ultrasonic energy reflected from each focal point, P, located at successive ranges, R, along the ultrasonic beam oriented at the angle, θ.

Under the direction of the digital controller 212, the receiver 208 provides delays during the scan such that the steering of the receiver 208 tracks with the direction of the beam steered by the transmitter 206, and such that the receiver 208 samples the echo signals at a succession of ranges, R, and provides the proper delays to dynamically focus at points, P, along the beam. Thus, each emission of an ultrasonic pulse results in the acquisition of a series of data points that represent the amount of reflected sound from a corresponding series of points, P, located along the ultrasonic beam.

In some configurations a physical device, such as a cone, may be used to steer the ultrasound beam to the target location. It will be appreciated by one skilled in the art that other options for directing ultrasound to a target location may be used with the present disclosure.

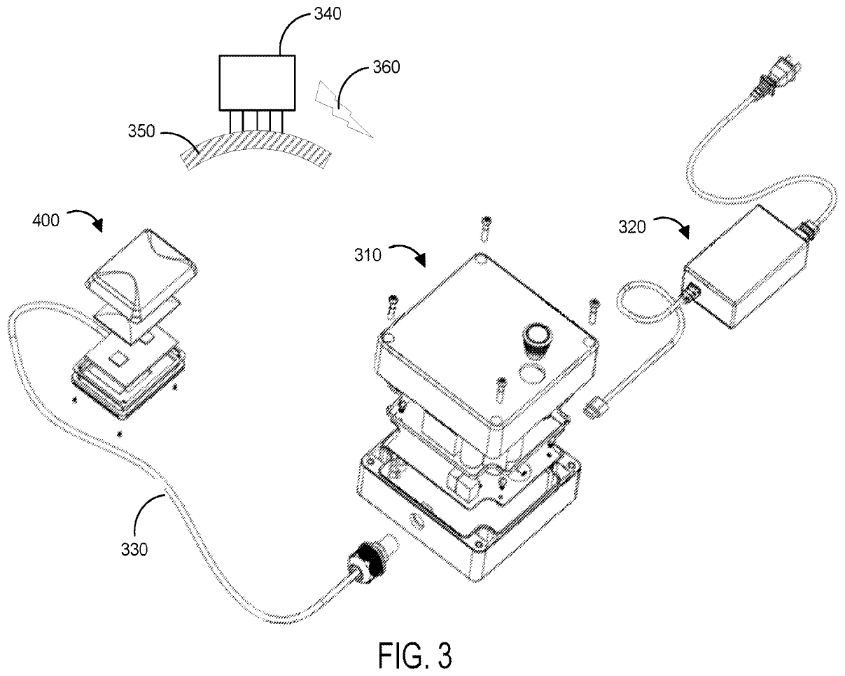

Referring to FIG. 3, a block diagram of a non-limiting example device for providing stimulation is shown. A therapy module 400 may be included and is capable of providing ultrasound stimulation to a subject. A control module 310 may be used to control the therapy module 400. Control module 310 may include instructions stored for controlling the therapy module 400. Biomarker feedback sensor 340 may provide for feedback data, such as a measure of cytokine levels, and may be in contact with a subject such as through skin layer 350. Biomarker feedback sensor 340 may be in wireless communication 360 with control module 310. Power for the system may be provided by power supply/adapter 320. Power supply/adapter 320 may be an AC power adapter that plugs into the wall for standard power input. The therapy module 400 may be wired to the control module 310 using cable 330. Alternatively, the therapy module 400 may be in wireless communication with control module 310.

The control module may be used for device operation and power transformation to the transmitting transducer.

In a non-limiting example, the control module 310 may generate +/−90V and 20V DC outputs. These DC outputs are supplied to the therapy module 400 to power beam forming ICs, MCU and other secondary components. Based on pre-programmed parameters, the beam forming ICs may generate tone-burst pulses with maximum Peak to Peak voltage of approximately 180V (+/−90V). Using these tone-burst pulses, the phase array transducer may activate piezoelectric elements (such as 128 piezoelectric elements) in a pre-defined manner and produces ultrasound pressure waves. For each device, the ultrasound pressure outputs may be measured and tuned to ensured that the appropriate intensities are below thresholds set by FDA guidance.

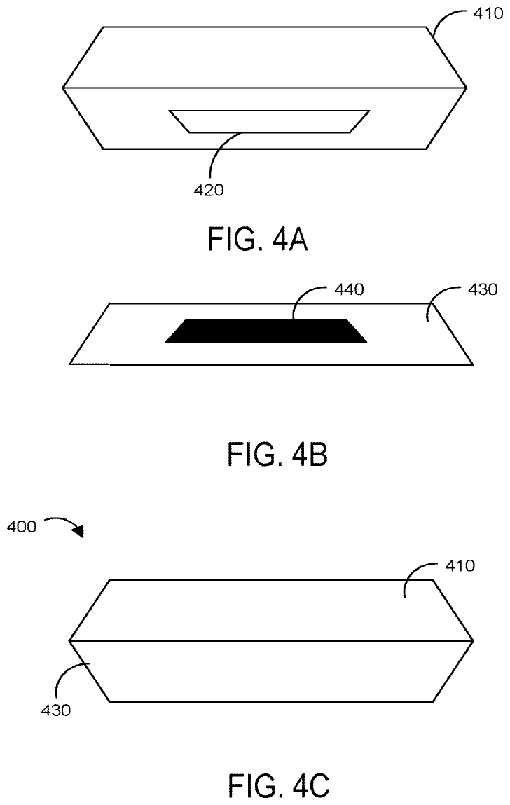

Referring to FIGS. 4A-4C, a non-limiting example therapy module 400 is shown in greater detail. Referring to FIG. 4A, the therapy module 400 may include an outer housing 410, and a transmission array 420. Referring to FIG. 4B, a base 430 may include a cut-out portion 440 for accommodating the transmission array 420. Referring to FIG. 4C, a profile view of therapy module 400 is shown assembled with outer housing 410 and base 430. In some configurations, therapy module 400 may be a small, lightweight, wearable component. In a non-limiting example, the therapy module 400 houses the beam forming integrated circuits (ICs), phase array ultrasonic transducers, and other auxiliary circuits (Bluetooth, memory, and the like).

The therapy module 400 may be adhered to the body of a subject with a disposable adhesive coupling pad. In a non-limiting example placement, the therapy module 400 may be placed on the torso of a subject.

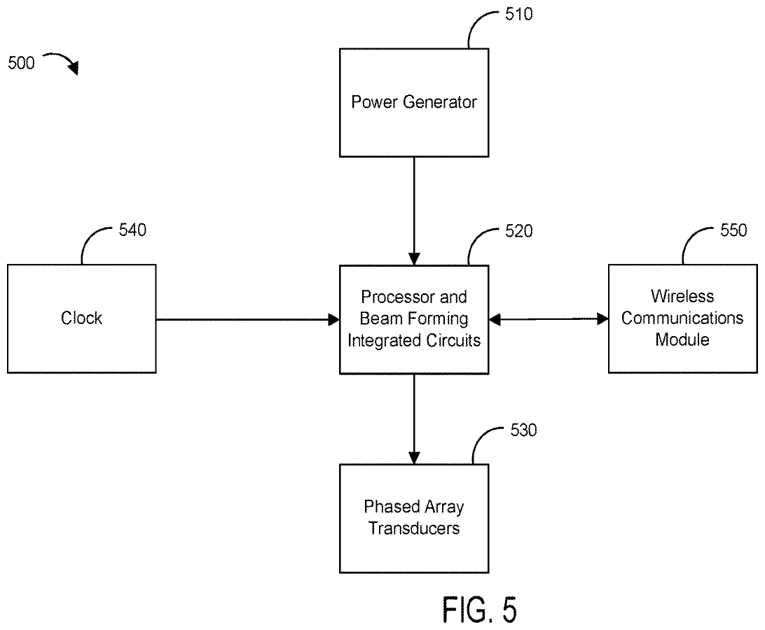

Referring to FIG. 5, a block diagram is shown for a non-limiting example system configuration 500. A power generator 510, which in some configurations may be a DC power supply, may provide power for processor and beam forming integrated circuits 520, which control a stimulation delivered by phased array transducers 530. A clock generator 540 may provide a signal for triggering the beam forming integrated circuits 520. A wireless communication module 550 may be coupled to the beam forming integrated circuits 520, and may provide for diagnostic access, data input/output, user interface access, and the like.

The system interface may allow for toggling of select features by an authorized user, such as a designated clinician or nurse, for administrate control via a custom tablet app. During therapeutic operation, use of the device may be limited to on/off operation with an automatic shutoff feature and may not require the use of a separate tablet device. Internal to the device, phasing algorithms may be provided for individual channel control to allow focusing and steering of the ultrasonic beam. This may allow the ultrasonic beam to be optimized for high intensity and pressure at the target site of the spleen. Additional parameters include the pulse duration and repetition frequency, the values of which may be optimized to achieve the desired therapeutic effect.

In one configuration, a system may be configured with 128-channel or 256-channel beam forming capability for high resolution steering and focusing. Peak frequency may be 400 to 600 kHz and channel spacing at 1.5 mm, which is equivalent to less than half of the ultrasonic wavelength (λ/2) in water at the 500 kHz ultrasonic frequency, optimizing the ability to steer and focus the ultrasound beam by avoiding energy loss due to side lobe generation. An ultra-compact design may be used with the wearable system being smaller than a standard business card.

In one configuration, a transducer for the system may include 128 transducer elements. Each element may be electrically and physically isolated, allowing for individual phasing and increasing the efficiency by nearly eliminating all acoustic cross-talk between channels. The 128-element array may be separated into four identical sections of 32 elements each. Each of the 32 elements in its respective section may be controlled in tandem with the three equivalent elements from the other sections. This allows the system to effectively operate in an equivalent manner as a 128-channel system for center beam focusing for spleen stimulation.

In one configuration, a transducer may include 32 channels tied to 32 elements for full steering and phasing in both X and Y axes. The transducer system can produce over 1 MPa of peak negative acoustic pressure and deliver energy to standard spleen depths, which commonly range from 2 to 6 cm. In some configurations, transducers may be provided with as high as 512 elements and channels for higher Pressure and larger acoustic ranges.

For spleen targeting, the systems have the ability to steer and focus the beam as necessary to achieve the desired therapeutic effect. In one configuration, by phasing the 64 channels of the system and transducer, the beam is able to steer efficiently towards the desired location. For the wearable device, a large number of programs may be stored into the internal memory for very fast rastering between steered and focused conditions. The phasing conditions may be preprogrammed based upon both the achievable targets of the ultrasonic device as well as the desired targets for therapy. In the case of spleen stimulation, the device may be configured for targeting a wide range of people with varying spleen depths. For certain therapies this can include hundreds or thousands of phasing algorithms which may be used in a raster pattern (e.g., switched) very quickly, possibly hundreds of programs within 1 or a few seconds.

The phased array transducer can be designed to produce a customized ultrasonic beam profile with optimum energy or pressure profile (i.e. peak pressure, depth, beam area etc.).

The electrical system may include multi-channel beam forming microprocessor chips for 32 individual channel control for phasing and focusing at +/−90V excitation. The wearable device may be wired to a power adapter which can accommodate standard electrical power inputs (i.e. 100-240V, 50/60 Hz).

In some configurations, the system includes a small wearable form factor, low cost, and amenability to scalable manufacturing. Existing instruments used for focused ultrasound medical applications are significantly more expensive; standard multi-channel systems routinely cost between $50,000 to $200,000 per unit, with large cart-based or desktop form factors. Conventional wearable ultrasound energy emission devices are designed for other purposes, such as wound-healing, and operate at inappropriate ultrasound frequencies or with only single or few channel operation, incapable of adequate beamforming for focusing. Hand-held medical ultrasound devices are designed for imaging purposes and are also inadequate for the energy delivery requirements of stimulating specific targets in the body or spleen stimulation, due to the linear array design which can beam form in only one axis or the tendency to operate at high frequencies, such as more than 3 to 10 MHz.

In some configurations, hybrid systems and transducers may be provided for ultrasonic imaging feedback. As targeting of nerves, organs, and obstructions is performed, a beam steering mechanism to avoid artifacts in a resulting image can be used in the process.

Non-Limiting Example Feedback Mechanisms Using Biomarker Sensors

The systems and methods in accordance with the present disclosure may be configured to use feedback provided by biomarker sensors, some of which provide immediate or near-term information regarding cytokine levels which may be relevant to the therapy delivered by the system.

A non-limiting example biomarker sensor includes a portable biosensor system capable of detecting cytokine IL-6 or other cytokines in unprocessed whole blood using filter paper-based immunosensors and smartphone imaging. This biosensor may detect small variations in IL-6 levels in under 20 minutes. The paper-based immunosensor generates a colorimetric signal and the pixel intensity of the colorimetric signal may be evaluated using the real-time densitometry enabled by the user-guided smartphone application. The biosensor could also be electrochemical aptamer-based sensing platform which measures concentrations of specific molecules directly in blood and even in the living body, or could also be label-free electrochemical impedance immunosensing platform with various types of electrodes. The ultrasound stimulation system can be controlled via Bluetooth using a smartphone app which could also receive information from this biosensing app for IL-6 and other cytokines that is housed on the same smartphone device; in some embodiments the biosensing app may be integrated into the ultrasound stimulation system app. Levels of ultrasound stimulation could be adjusted in response to detected increases or decreases in cytokines such as IL-6 based on a predetermined algorithm and thresholds, incorporating data from past hours or days of cytokine monitoring.

A non-limiting example biomarker sensor includes a miniaturized sensor that uses a fluid such as saliva or sweat to measure cytokine or other biomarkers relevant to inflammation levels. These devices may use various technologies, such as light waves (i.e. ultraviolet or infrared).

Communication with the system may be provided in real-time or delayed using the communication protocols Bluetooth, RFID, near-field, or WiFi. In some configurations the biomarker sensors may be physically detached from the rest of the system while communicating wirelessly. Direct integration of a biomarker sensor into the system may also be provided. In this configuration the biomarker sensor may be integrated directly into the device.

Communication to the system via an intermediary system may also be provided, such as a networked server. The biomarker sensor may be internet-enabled and provide data to a managed server which will then push the data back through the internet to system, which may also be networked accordingly. The data may be processed by the intermediary server or passed to the system directly.

Clinical applications for the systems and methods according to the present disclosure include treatment of autoimmune disorder, and inflammatory conditions, such as rheumatoid arthritis, and the like. Other inflammatory and autoimmune disorders may be treated in which IL-6, IL-10 and TNF-α may be modulated.

Referring to FIG. 7, non-limiting example results are shown for a randomized, controlled, blinded clinical trial in rheumatoid arthritis patients in which one group of patients received ultrasound stimulation of the spleen and another group receives a sham condition where no energy is transmitted from the transducer. As depicted, ultrasound stimulation of the spleen in an inflammation disorder can modulate cytokines and erythrocyte sedimentation rate (ESR; a standard measure of inflammation in the body) in response to ultrasound of the spleen. The ESR values shows how the patients who receive no treatment experience an increase in inflammation over time (treatment is for 14 days with a 1-week no-treatment follow-up phase), whereas the stimulated group has a flatter ESR curve. TNF-alpha, IL-6 and IL-10 were the biomarkers that could be decreased with treatment that was not observed for the sham group. Patients who received ultrasound treatment also exhibited a reduction in the number of swollen joints. Ultrasound stimulation of the spleen can modulate the immune response in human patients with inflammatory/autoimmune disorders, which can be applied to stimulating other end-organs, lymph-nodes or nerve pathways involved with the immune response, as well as for treating other inflammatory tissue or neural injuries such as acute kidney failure, stroke, and spinal cord injury.

The present disclosure has described one or more preferred embodiments, and it should be appreciated that many equivalents, alternatives, variations, and modifications, aside from those expressly stated, are possible and within the scope of the invention.

What is claimed is:

1. A method for ultrasound stimulation of a subject, comprising:
applying ultrasound stimulation to a target of the subject based on initial ultrasound stimulation parameters;
receiving feedback data after the stimulation has been applied, wherein the feedback data are indicative of biomarkers being modulated by the applied ultrasound stimulation;
adjusting the initial ultrasound stimulation parameters based upon the received feedback data to create adjusted ultrasound stimulation parameters; and
applying ultrasound stimulation to the target of the subject based on the adjusted ultrasound stimulation parameters to treat an inflammatory condition of the subject.

2. The method of claim 1, wherein the feedback data is received between 6 hours up to and including 14 days after the stimulation.

3. The method of claim 1, wherein the feedback data is received between 3.5 days up to and including 14 days after the stimulation.

4. The method of claim 1, wherein the feedback data is received between 3.5 days up to and including 7 days after the stimulation.

5. The method of claim 1, wherein the feedback data includes at least one of a ratio or a combination of two or more biomarkers.

6. The method of claim 5, wherein the ratio of biomarkers includes a ratio of at least one of interleukin (IL) IL-6 to IL-10, tumor necrosis factor (TNF) TNF-α to erythrocyte sedimentation rate (ESR), or IL-6 to TNF-α.

7. The method of claim 1, wherein the feedback data is received by at least one of a sensor or a blood draw.

8. The method of claim 7, wherein the sensor includes a cytokine sensor.

9. The method of claim 8, wherein the cytokine sensor is implanted in the subject.

10. The method of claim 1, wherein the biomarkers include at least one of ESR, TNF-α, IL-6, IL-10, or a combination thereof.

11. The method of claim 1, wherein the target is the spleen.

12. The method of claim 1, wherein the initial ultrasound stimulation parameters includes at least one of stimulation in the range of greater than or equal to 500 kHz to less than or equal to 1 MHz; pressures in a range of greater than or equal to 100 kPa to less than or equal to 500 kPa; or duty cycles of greater than or equal to 16% to less than or equal to 50%.

13. The method of claim 1, wherein the adjusted ultrasound stimulation parameters includes at least one of stimulation in the range of greater than or equal to 100 kHz to less than or equal to 7 MHz; pressures in a range of greater than or equal to 25 kPa to less than or equal to 10 MPa; stimulation on time of greater than or equal to 50 microseconds to less than or equal to 2 seconds; or stimulation off time of greater than or equal to 1 ms to less than or equal to 10 s.

14. The method of claim 1, wherein at least one of the initial ultrasound stimulation parameters or the adjusted ultrasound stimulation parameters includes stimulation of 1 MHz.

15. The method of claim 1, wherein at least one of the initial ultrasound stimulation parameters or the adjusted ultrasound stimulation parameters includes a pressure of 350 kPa.

16. The method of claim 1, wherein adjusting the initial ultrasound stimulation parameters includes stopping stimulation when the feedback data indicates at least one of the biomarkers has reached a plateau.

17. The method of claim 16, wherein the plateau occurs at greater than or equal to 3.5 days and less than or equal to 14 days after initial ultrasound stimulation.

18. The method of claim 1, wherein adjusting the initial ultrasound stimulation parameters includes adjusting at least one of a duty cycle, a duration, a number of bursts, a length of a burst, a frequency, a pressure, or a beamforming target of the ultrasound stimulation.

19. The method of claim 18, wherein adjusting the duration of the ultrasound stimulation includes applying the ultrasound stimulation for up to and including 30 minutes.

20. A system for ultrasound stimulation of a subject, comprising:
a feedback sensor implanted within a subject;
a computer system configured to:
i) apply ultrasound stimulation to a target of the subject with initial ultrasound stimulation parameters;
ii) receive feedback data from a sensor, wherein the feedback data are measured by the sensor and are indicative of biomarkers being modulated by the applied ultrasound stimulation;
iii) adjust the initial ultrasound stimulation parameters based upon the received feedback data to create adjusted ultrasound stimulation parameters; and
iv) apply ultrasound stimulation to the target of the subject with the adjusted ultrasound stimulation parameters to treat an inflammatory condition of the subject.

21. The system of claim 20, wherein the computer system is further configured to receive feedback data between 6 hours up to and including 14 days after the stimulation.

22. The system of claim 20, wherein the computer system is further configured to receive feedback data between 3.5 days up to and including 14 days after the stimulation.

23. The system of claim 20, wherein the computer system is further configured to receive feedback data between 3.5 days up to and including 7 days after the stimulation.

24. The system of claim 20, wherein the feedback data includes at least one of a ratio or a combination of two or more biomarkers.

25. The system of claim 24, wherein the ratio of biomarkers includes a ratio of at least one of interleukin (IL) IL-6 to IL-10, tumor necrosis factor (TNF) TNF-α to erythrocyte sedimentation rate (ESR), or IL-6 to TNF-α.

26. The system of claim 20, wherein the sensor is a cytokine sensor implanted in the subject.

27. The system of claim 20, wherein the biomarkers include at least one of ESR, TNF-α, IL-6, IL-10, or a combination thereof.

28. The system of claim 20, wherein the target is the spleen.

29. The system of claim 20, wherein the initial ultrasound stimulation parameters includes at least one of stimulation in the range of greater than or equal to 500 kHz to less than or equal to 1 MHz; pressures in a range of greater than or equal to 100 kPa to less than or equal to 500 kPa; or duty cycles of greater than or equal to 16% to less than or equal to 50%.

30. The system of claim 20, wherein the adjusted ultrasound stimulation parameters includes at least one of stimulation in the range of greater than or equal to 100 kHz to less than or equal to 7 MHz; pressures in a range of greater than or equal to 25 kPa to less than or equal to 10 MPa; stimulation on time of greater than or equal to 50 microseconds to less than or equal to 2 seconds; or stimulation off time of greater than or equal to 1 ms to less than or equal to 10 s.

31. The system of claim 20, wherein at least one of the initial ultrasound stimulation parameters or the adjusted ultrasound stimulation parameters includes stimulation of 1 MHz.

32. The system of claim 20, wherein at least one of the initial ultrasound stimulation parameters or the adjusted ultrasound stimulation parameters includes a pressure of 350 kPa.

33. The system of claim 20, wherein the computer system is further configured to stop stimulation when the feedback data indicates at least one of the biomarkers has reached a plateau.

34. The system of claim 33, wherein the plateau occurs at greater than or equal to 3.5 days and less than or equal to 14 days after initial ultrasound stimulation.

35. The system of claim 20, wherein the computer system is further configured to adjust the initial ultrasound stimulation parameters by adjusting at least one of a duty cycle, a duration, a number of bursts, a length of a burst, a frequency, a pressure, or a beamforming target of the ultrasound stimulation.

36. The system of claim 35, wherein the computer system is further configured to adjust the duration of the ultrasound stimulation by applying the ultrasound stimulation for up to and including 30 minutes.

37. A method for ultrasound stimulation of a subject, comprising:
determining a target for the ultrasound stimulation of the subject;
determining ultrasound stimulation parameters;
applying ultrasound stimulation to the target of the subject with the ultrasound stimulation parameters to treat an inflammatory condition of the subject, wherein biomarkers are modulated in the treatment and include a combination of at least two of: erythrocyte sedimentation rate (ESR), tumor necrosis factor TNF-α, interleukin IL-6, or interleukin IL-10.

38. The method of claim 37, wherein the biomarkers are modulated by determining a ratio of biomarkers, including a ratio of at least one of IL-6 to IL-10, TNF-α to ESR, or IL-6 to TNF-α.

39. The method of claim 37, wherein the target is the spleen.

40. The method of claim 37, wherein the ultrasound stimulation parameters includes at least one of stimulation in the range of greater than or equal to 100 kHz to less than or equal to 7 MHz; pressures in a range of greater than or equal to 25 kPa to less than or equal to 10 MPa; or duty cycles of greater than or equal to 16% to less than or equal to 50%.

41. The method of claim 37, wherein the ultrasound stimulation parameters includes stimulation of 1 MHz.

42. The method of claim 37, wherein the ultrasound stimulation parameters includes pressure of 350 kPa.
